(19) United States
(12) Patent Application Publication
(10) Pub. No.: US 2004/0249220 A1
(43) Pub. Date: Dec. 9, 2004

(54) Process for producing optically active 2-alkoxy-1-(trifluoromethyl-substituted phenyl) ethanol derivatives

(76) Inventors: Akihiro Ishii, Saitama (JP); Masatomi Kanai, Saitama (JP); Yokusu Kuriyama, Saitama (JP); Manabu Yasumoto, Saitama (JP); Kenjin Inomiya, Saitama (JP); Takashi Ootsuka, Saitama (JP); Koji Ueda, Saitama (JP)

Correspondence Address: CROWELL & MORING LLP, INTELLECTUAL PROPERTY GROUP, P.O. BOX 14300, WASHINGTON, DC, 20044-4300, US

(21) Appl. No.: 10/481,620

(22) PCT Filed: Oct. 22, 2003
(86) PCT No.: PCT/JP03/13474

(30) Foreign Application Priority Data
Oct. 24, 2002 (JP) 2003 310180

(51) Int. Cl. C07C 41/01

(52) U.S. Cl. 568/662

(57) ABSTRACT

A process for producing an optically active 2-alkoxy-1-(trifluoromethyl-substituted phenyl)ethanol derivative represented by the formula 5:

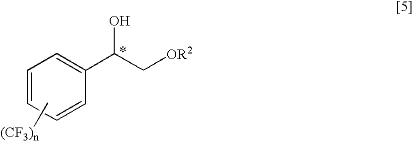

where R² represents a C₁-C₆ lower alkyl group, n represents 1 or 2 and * represents a chiral carbon, includes the steps of (a) reducing an optically active, hydroxyl-protective, trifluoromethyl-substituted mandelate represented by the formula 1, by a hydride reducing agent, into an optically active, hydroxyl-protective 2-hydroxy-1-(trifluoromethyl-substituted phenyl)ethanol represented by the formula 2; (b) reacting the hydroxyl-protective hydroxyethanol represented by the formula 2, with an alkylation agent represented by the formula 3, in the presence of a base, thereby producing an optically active, hydroxyl-protective 2-alkoxy-1-(trifluoromethyl-substituted phenyl)ethanol represented by the formula 4; and (c) deprotecting the hydroxyl-protective alkoxyethanol represented by the formula 4 into the alkoxyethanol derivative represented by the formula 5.

### BACKGROUND OF THE INVENTION

The present invention relates to a process for producing optically active 2-alkoxy-1-(trifluoromethyl-substituted phenyl)ethanol derivatives, which are important intermediates for medicines and agricultural chemicals.

Of these derivatives, racemic 2-methoxy-1-(4′-trifluoromethylphenyl)ethanol has been reported as being an important intermediate for medicinal candidate compounds having anti-HIV activity (see U.S. Pat. No. 6,391,865 and International Application WO 00/66558). This methoxyethanol is produced by epoxidating 4-trifluoromethylstyrene by m-chloroperbenzoic acid (m-CPBA), followed by an end-ring opening with sodium methoxide. This production process may not be a good industrial production process, since the raw material is not easily available and since the yield is not sufficiently high. According to these publications, a racemic 2-methoxy-1-(4′-trifluoromethylphenyl)ethanol is mesylated, followed by condensation with an optically active 2-methylpiperazine derivative. The resulting mixture of two different diastereomers (1:1) are separated by column chromatography. Then, only the necessary diastereomer is used for inducing medicinal candidate compounds having anti-HIV activity. This process for producing the necessary diastereomer is not suitable for a mass production due to an excessive load. Furthermore, it is natural that the yield of the target diastereomer produced from a racemic 2-methoxy-1-(4′-trifluoromethylphenyl)ethanol never exceeds 50% due to no use of the unnecessary diastereomer.

Although optically active 4-trifluoromethylmandelic acid, (R)-3-trifluoromethylmandelic acid, and optically active 3,5-bis(trifluoromethyl)mandelic acid are known (see J. Med. Chem. (1974) 17, 1, p. 34-41; Proc. Natl. Acad. Sci. (1997) 94, 18, p. 9590-9595; WO 93/10074; European Patent Applications 0052963 and 0040000; and Chirality, 1999, 11, 5/6, p. 420-425), other optically active trifluoromethyl-substituted mandelic acids are not known.

J. Am. Chem. Soc., 2002, 124, 12, p. 2870-2871 discloses ethyl-(R)-4-trifluoromethylmandelate.

### SUMMARY OF THE INVENTION

It is therefore an object of the present invention to provide a process for industrially producing optically active 2-alkoxy-1-(trifluoromethyl-substituted phenyl)ethanol derivatives, which are important intermediates for medicines and agricultural chemicals, with high optical purity and high yield, from a raw material that is easily available in an industrial scale.

According to the present invention, there is provided a first process for producing an optically active 2-alkoxy-1-(trifluoromethyl-substituted phenyl)ethanol derivative represented by the formula 5:

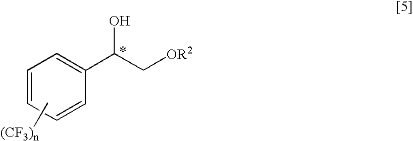

where R² represents a lower alkyl group having a carbon atom number of 1-6, n represents an integer of 1 or 2, and * represents a chiral carbon. The first process comprises the steps of:

(a) reducing an optically active, hydroxyl-protective, trifluoromethyl-substituted mandelate represented by the formula 1, by a hydride reducing agent,

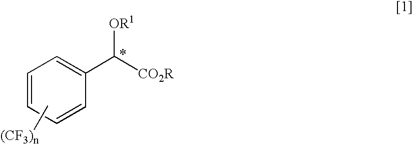

where R represents a lower alkyl group having a carbon atom number of 1-6, R¹ represents a protecting group for hydroxyl group, and n and * are defined as in the formula 5, into an optically active, hydroxyl-protective, 2-hydroxy-1-(trifluoromethyl-substituted phenyl)ethanol represented by the formula 2:

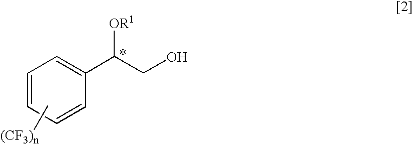

where R¹ is defined as in the formula 1, and n and * are defined as in the formula 5,

(b) reacting the hydroxyl-protective hydroxyethanol represented by the formula 2, with an alkylation agent represented by the formula 3, in the presence of a base,

R²—X [3]

where R² is defined as in the formula 5, and X represents a leaving group, thereby producing an optically active, hydroxyl-protective 2-alkoxy-1-(trifluoromethyl-substituted phenyl)ethanol represented by the formula 4:

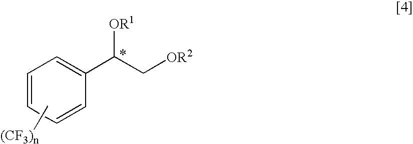

where R¹ is defined as in the formula 1, and R², n and * are defined as in the formula 5, and

(c) deprotecting the hydroxyl-protective alkoxyethanol represented by the formula 4 into the alkoxyethanol derivative represented by the formula 5.

The first process may be a second process for producing an optically active 2-methoxy-1-(4′-trifluoromethylphenyl)ethanol represented by the formula 10:

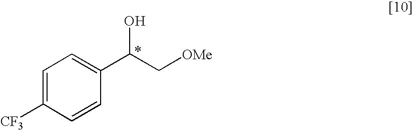

where Me represents a methyl group and * represents a chiral carbon. The second process comprises the steps of:

(a) reducing an optically active, hydroxyl-protective 4-trifluoromethylmandelate represented by the formula 6, by sodium borohydride,

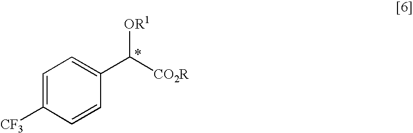

where R represents a lower alkyl group having a carbon atom number of 1-6, R¹ represents a protecting group for hydroxyl group, and * is defined as in the formula 10, into an optically active, hydroxyl-protective 2-hydroxy-1-(4′-trifluoromethylphenyl)ethanol represented by the formula 7:

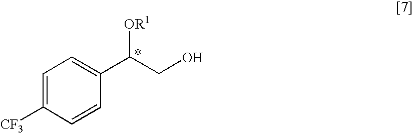

where R¹ is defined as in the formula 6, and * is defined as in the formula 10,

(b) reacting the hydroxyl-protective hydroxyethanol represented by the formula 7, with a methylation agent represented by the formula 8, in the presence of a base,

Me-X [8]

where Me represents a methyl group, and X represents a leaving group, thereby producing an optically active, hydroxyl-protective 2-methoxy-1-(4′-trifluoromethylphenyl)ethanol represented by the formula 9:

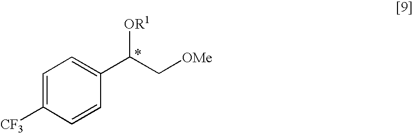

where R¹ is defined as in the formula 6, and Me and * are defined as in the formula 10, and

(c) deprotecting the hydroxyl-protective methoxyethanol represented by the formula 9 into the methoxyethanol represented by the formula 10.

As stated above, the steps (a), (b) and (c) of the second process respectively correspond to those of the first process.

According to the present invention, it is optional to produce an optically active, hydroxyl-protective, trifluoromethyl-substituted mandelate represented by the formula 1 (i.e., the raw material of the step (a) of the first process) by a third process. The third process comprises the steps of:

(d) reacting an optically active trifluoromethyl-substituted mandelic acid represented by the formula 11, with a lower alcohol having a carbon atom number of 1-6 in the presence of an acid catalyst,

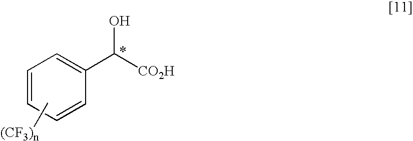

where n and * are defined as in the formula 5, thereby producing an optically active trifluoromethyl-substituted mandelate represented by the formula 12:

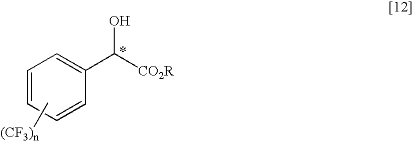

where R is defined as the formula 1, and n and * are defined as in the formula 5, and

(e) protecting a hydroxyl group of the mandelate represented by the formula 12, thereby producing the hydroxyl-protective mandelate represented by the formula 1.

According to the present invention, the third process may be a fourth process for producing an optically active, hydroxyl-protective 4-trifluoromethylmandelate represented by the formula 6 (i.e., the raw material of the step (a) of the second process). The fourth process comprises the steps of:

(d) reacting an optically active 4-trifluoromethylmandelic acid represented by the formula 13, with a lower alcohol having a carbon atom number of 1-6 in the presence of an acid catalyst,

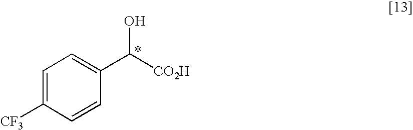

where * is defined as in the formula 10, thereby producing an optically active 4-trifluoromethylmandelate represented by the formula 14:

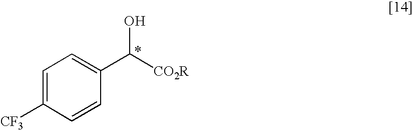

where R is defined as the formula 6, and * is defined as in the formula 10, and

(e) protecting a hydroxyl group of the mandelate represented by the formula 14, thereby producing the hydroxyl-protective mandelate represented by the formula 6.

As stated above, the steps (d) and (e) of the fourth process respectively correspond to those of the third process.

According to the present invention, the above optically active trifluoromethyl-substituted mandelate represented by the formula 12 may be an optically active methyl trifluoromethyl-substituted mandelate represented by the formula 15:

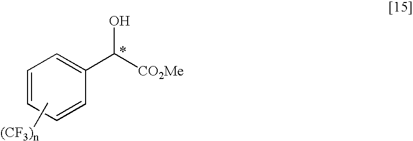

where Me represents a methyl group, n and * are defined as in the formula 5.

According to the present invention, the above optically active 4-trifluoromethylmandelate represented by the formula 14 may be an optically active methyl-4-trifluoromethylmandelate represented by the formula 16:

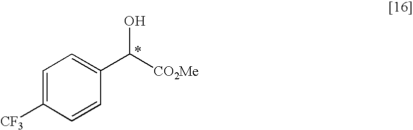

where Me represents a methyl group, and * is defined as in the formula 10.

As stated above, it is optional to combine the first process with the third process or to combine the second process with the fourth process to sequentially conduct the five steps (d), (e), (a), (b), and (c), thereby producing the target product, the alkoxyethanol of the formula 5, from the optically active mandelic acid of the formula 11.

### DESCRIPTION OF THE PREFERRED EMBODIMENTS

According to any one of the first to fourth processes of the present invention, it is possible to maintain high optical purity of the raw material through each step. Furthermore, it is possible to conduct the reaction of each step with high selectivity under a mild reaction condition, while by-products that are difficult for separation are almost not produced. Thus, it is possible to industrially produce the target product (i.e., an optically active 2-alkoxy-1-(trifluoromethyl-substituted phenyl)ethanol derivative represented by the formula 5) with high optical purity and high chemical purity by the first or second process, with or without a combination of the first or second process with the third or fourth process. This combination is very useful for producing the target product, since the raw material (an optically active trifluoromethyl-substituted mandelic acid) of the step (d) of the third or fourth process is easily available with high optical purity.

As stated above, it is possible to produce the target product (i.e., an optically active 2-alkoxy-1-(trifluoromethyl-substituted phenyl)ethanol derivative of the formula 5) by combining the first or second process with the third or fourth process to sequentially conduct the steps (d), (e), (a), (b), and (c), as shown by the following scheme.

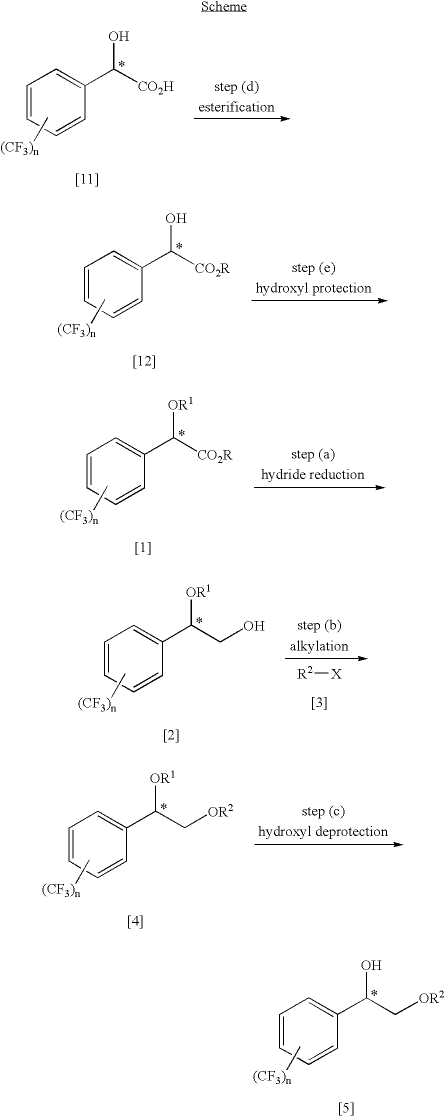

Of intermediates in the above scheme, an optically active ethyl 4-trifluoromethyl mandelate (corresponding to the formula [12]) is a known compound. However, in view of this ethyl mandelate as an intermediate for producing the target product of the present invention, it may not be preferable to produce the ethyl mandelate by the step (d) using ethanol, which is cumbersome in handling and management in terms of tax rule. Furthermore, in view of purification through distillation, it is preferable to use an alkyl ester (mandelate) that has a boiling point as low as possible, as an intermediate for producing the target product. The present inventors unexpectedly found that the target product can desirably be produced by using particularly methanol as the lower alcohol of the step (d). In this case, an optically active methyl trifluoromethyl-substituted mandelate is produced as an intermediate.

The step (d), esterification, is described in detail as follows. It is possible to conduct the step (d) by reacting an optically active trifluoromethyl-substituted mandelic acid represented by the formula 11, with a C₁-C₆ lower alcohol in the presence of an acid catalyst.

Examples of this mandelic acid of the formula 11 are (R)-2-trifluoromethylmandelic acid, (S)-2-trifluoromethylmandelic acid, (R)-3-trifluoromethylmandelic acid, (S)-3-trifluoromethylmandelic acid, (R)-4-trifluoromethylmandelic acid, (S)-4-trifluoromethylmandelic acid, (R)-2,3-bis(trifluoromethyl)mandelic acid, (S)-2,3-bis(trifluoromethyl)mandelic acid, (R)-2,4-bis(trifluoromethyl)mandelic acid, (S)-2,4-bis(trifluoromethyl)mandelic acid, (R)-2,5-bis(trifluoromethyl)mandelic acid, (S)-2,5-bis(trifluoromethyl)mandelic acid, (R)-2,6-bis(trifluoromethyl)mandelic acid, (S)-2,6-bis(trifluoromethyl)mandelic acid, (R)-3,4-bis(trifluoromethyl)mandelic acid, (S)-3,4-bis(trifluoromethyl)mandelic acid, (R)-3,5-bis(trifluoromethyl)mandelic acid, and (S)-2,5-bis(trifluoromethyl)mandelic acid. Although these examples contain novel compounds, such novel compounds can be produced by using substrates having a trifluoromethyl group(s) on a desired position(s) of the phenyl group, in view of the disclosures of J. Med. Chem. (1974) 17, 1, p. 34-41; Proc. Natl. Acad. Sci. (1997) 94, 18, p. 9590-9595; WO 93/10074; European Patent Applications 0052963 and 0040000; and Chirality, 1999, 11, 5/6, p. 420-425.

Examples of the lower alcohol used in the step (d) include methanol, ethanol, n-propanol, i-propanol, n-butanol, 2-butanol, n-pentanol, n-hexanol, and cyclohexanol. Of these, methanol, n-propanol and i-propanol are preferable, and methanol is more preferable.

In the step (d), the lower alcohol may be used in an amount of at least one equivalent per equivalent of the mandelic acid of the formula 11. In particular, the lower alcohol can be used excessively as a reaction solvent.

Examples of the acid catalyst used in the step (d) include organic acids (e.g., benzenesulfonic acid, p-toluenesulfonic acid, and 10-camphorsulfonic acid) and inorganic acids (e.g., hydrochloric acid, hydrobromic acid, sulfuric acid, phosphoric acid, zinc chloride, and titanium tetrachloride). Of these, p-toluenesulfonic acid, sulfuric acid, and zinc chloride are preferable. In particular, sulfuric acid is more preferable.

The acid catalyst of the step (d) may be in a catalytic amount, preferably 0.001-0.9 equivalents, more preferably 0.001-0.5 equivalents, per equivalent of the mandelic acid of the formula 11.

As the reaction of the step (d) proceeds, water is produced as a by-product. Thus, it is possible to conduct the step (d) under a dehydration condition in order to accelerate the reaction. The way of this dehydration is not particularly limited. It is preferable to use a dehydration agent such as zeolite (trade name: molecular sieve), phosphorus pentoxide, anhydrous sodium sulfate, and anhydrous magnesium sulfate. Furthermore, in case that the lower alcohol used in the step (d) is immiscible with water and is lower than water in specific gravity, and forms an azeotropic mixture with water, it is preferable to conduct (a) a first dehydration in which water is removed from a Dean-Stark tube under reflux condition or (b) a second dehydration in which water is removed from a Dean-Stark tube under reflux condition using a reaction solvent such as benzene or toluene.

The reaction temperature of the step (d) may be from 0° C. to +200° C., preferably from 0° C. to +150° C., more preferably from 0° C. to +100° C.

Although the reaction time of the step (d) may be 72 hr or shorter, it may be varied depending on the type of the substrate and the reaction conditions. Therefore, it is preferable to terminate the reaction after confirming that the raw material has almost been consumed by checking the progress of the reaction by an analytical means such as gas chromatography, thin layer chromatography, liquid chromatography, and NMR.

After the reaction of the step (d), it is possible to obtain a crude product by conducting a normal post-treatment. According to need, the crude product may be subjected to purification such as activated carbon treatment, distillation, recrystallization, and column chromatography, thereby obtaining the target product, an optically active trifluoromethyl-substituted mandelate of the formula 12, with high chemical purity.

The step (e), hydroxyl-group protection, is described in detail as follows. It is possible to conduct the step (e) by protecting a hydroxyl group of the mandelate of the formula 12.

The protecting group (R¹) for the hydroxyl group is not limited to particular types. It may be chosen from those cited in Chapter Second (p. 17-245) of “Protective Groups in Organic Synthesis, Third Edition” written by Theodora W. Greene and Peter G. M. Wuts, published by Wiley-Interscience, New York (1999). Of those, preferable ones are tetrahydropyranyl group (THP), 1-ethoxyethyl group, methoxymethyl group, triphenylmethyl group, benzyl group, trimethylsilyl group, triethylsilyl group, t-butyldimethylsilyl group, and t-butyldiphenylsilyl group. More preferable ones are THP, methoxymethyl group, and triethylsilyl group.

The protecting groups (R¹) for the hydroxyl group can be classified into A-type and B-type, depending on the way of the protection. A-type protecting groups are introduced by the reaction with a protecting agent in the presence of an acid catalyst. In contrast, B-type protecting groups are introduced by the reaction with a protecting agent in the presence of a base. Examples of A-type protecting groups include THP and 1-ethoxyethyl group. Examples of B-type protecting groups include methoxymethyl group, triphenylmethyl group, benzyl group, trimethylsilyl group, triethylsilyl group, t-butyldimethylsilyl group, and t-butyldiphenylsilyl group. Examples of A-type protecting agents, corresponding to their respective A-type protecting groups, include dihydropyrane (DHP) and ethyl vinyl ether. Examples of B-type protecting agents, corresponding to their respective B-type protecting groups, include methoxymethyl chloride, triphenylmethyl chloride, benzyl bromide, trimethylsilyl chloride, triethylsilyl chloride, t-butyldimethylsilyl chloride, and t-butyldiphenylsilyl chloride.

The protecting agent may be in an amount of at least one equivalent, preferably 1-20 equivalents, more preferably 1-10 equivalents, per equivalent of the optically active trifluoromethyl-substituted mandelate of the formula 12.

The acid catalyst for A-type protecting agent may be chosen from p-toluenesulfonic acid, pyridinium p-toluenesulfonate (PPTS), SO₃H-type ion exchange resin, and hydrochloric acid. Of these, preferable ones are p-toluenesulfonic acid, PPTS, and hydrochloric acid. More preferable ones are p-toluenesulfonic acid and PPTS.

The acid catalyst for A-type protecting agent may be in a catalytic amount, preferably 0.001-0.9 equivalents, more preferably 0.001-0.5 equivalents, per equivalent of the mandelate of the formula 12.

The base for B-type protecting agent may be chosen from sodium hydride, potassium hydride, triethylamine, diisopropylethylamine, pyridine, 2,6- lutidine, 2,4,6-collidine, 4-N,N-dimethylaminopyridine, 1,1,1,3,3,3-hexamethyldisilazane, and imidazole. Of these, preferable ones are sodium hydride, triethylamine, diisopropylethylamine, pyridine, 2,6-lutidine, 4-N,N-dimethylaminopyridine, and imidazole. More preferable ones are sodium hydride, triethylamine, 4-N,N-dimethylaminopyridine, and imidazole.

The base for B-type protecting agent may be at least one equivalent, preferably 1-20 equivalents, more preferably 1-10 equivalents, per equivalent of the mandelate of the formula 12.

The reaction solvent for conducting the step (e) may be selected from aliphatic hydrocarbons (e.g., n-pentane, n-hexane, cyclohexane, and n-heptane), aromatic hydrocarbons (e.g., benzene, toluene, ethylbenzene, xylene, and mesitylene), halogenated hydrocarbons (e.g., methylene chloride, chloroform, carbon tetrachloride, and 1,2-dichloroethane), ethers (e.g., diethyl ether, tetrahydrofuran, t-butyl methyl ether, and 1,4-dioxane), esters (e.g., ethyl acetate, and n-butyl acetate), amides (e.g., hexamethylphosphoric triamide, N,N-dimethylformamide, N,N-dimethylacetamide, and N-methylpyrolidone), nitriles (e.g., acetonitrile and propionitrile), and dimethylsulfoxide. Of these, preferable ones are toluene, methylene chloride, 1,2-dichloroethane, tetrahydrofuran, t-butyl methyl ether, ethyl acetate, N,N-dimethylformamide, and acetonitrile. More preferable ones are toluene, methylene chloride, tetrahydrofuran, ethyl acetate, and N,N-dimethylformamide. The above-exemplified reaction solvents can be used alone or in combination.

The amount of the reaction solvent is not particularly limited. It may be at least one part by volume, preferably 1-50 parts by volume, more preferably 1-20 parts by volume, relative to one part by volume of the mandelate of the formula 12.

The reaction temperature of the step (e) may be from −30° C. to +200° C., preferably from −30° C. to +150° C., more preferably from −30° C. to +100° C.

Although the reaction time of the step (e) may be 72 hr or shorter, it may be varied depending on the type of the substrate and the reaction conditions. Therefore, it is preferable to terminate the reaction after confirming that the raw material has almost been consumed by checking the progress of the reaction by an analytical means such as gas chromatography, thin layer chromatography, liquid chromatography, and NMR.

After the reaction of the step (e), it is possible to obtain a crude product by conducting a normal post-treatment. According to need, the crude product may be subjected to purification such as activated carbon treatment, distillation, recrystallization, and column chromatography, thereby obtaining the target product, an optically active, hydroxyl-protective, trifluoromethyl-substituted mandelate of the formula 1, with high chemical purity.

The step (a), hydride reduction, is described in detail as follows. It is possible to conduct the step (a) by reducing the hydroxyl-protective mandelate of the formula 1 by a hydride reducing agent.

A hydride reducing agent to be used in the step (a) can be selected from (1) aluminium hydrides such as (i-Bu)₂AlH, (i-Bu)₃Al, [2,6-(t-Bu)₂-4-Me-Ph]Al(i-Bu)₂, LiAlH₄, LiAlH(OMe)₃, LiAlH(O-t-Bu)₃, and NaAlH₂(OCH₂CH₂OCH₃)₂; (2) boron hydrides such as diborane, BH₃.THF, BH₃.SMe₂, BH₃.NMe₃, 9-BBN, NaBH₄, NaBH₄-CeCl₃, LiBH₄, Zn(BH₄)₂, Ca(BH₄)₂, Li(n-Bu)BH₃, NaBH(OMe)₃, NaBH(OAc)₃, NaBH₃CN, Et₄NBH₄, Me₄NBH(OAc)₃, (n-Bu)₄NBH₃CN, (n-Bu)₄NBH(OAc)₃, Li(sec-Bu)₃BH, K(sec-Bu)₃BH, LiSia₃BH, KSia₃BH, LiEt₃BH, KPh₃BH, (Ph₃P)₂CuBH₄, ThxBH₂, Sia₂BH, catecholborane, IpcBH₂, and Ipc₂BH; and (3) silicon hydrides such as Et₃SiH, PhMe₂SiH, Ph₂SiH₂, and PhSiH₃-Mo(CO)₆, where Bu represents a butyl group, Ph represents a phenyl group, Me represents a methyl group, THF represents tetrahydrofuran, 9-BBN represents 9-borabicyclo[3.3.1]nonane, Ac represents an acetyl group, Sia represents a thiamyl group, Et represents an ethyl group, Thx represents a thexyl group, and Ipc represents an isopinocampheyl group. Of these, preferable examples are LiAlH₄, diborane, NaBH₄, and LiBH₄. In particular, NaBH₄ is more preferable. It is possible to use a combination of at least one of these hydrides and at least one of various inorganic salts.

In the step (a), the hydride reducing agent may be in an amount of 0.25 equivalents or greater, preferably 0.25-10 equivalents, more preferably 0.25-7 equivalents, per equivalent of the hydroxyl-protective mandelate of the formula 1.

A reaction solvent usable in the step (a) is not particularly limited. Its examples are (1) aliphatic hydrocarbons such as n-pentane, n-hexane, cyclohexane, and n-heptane; (2) aromatic hydrocarbons such as benzene, toluene, ethylbenzene, xylene, and mesitylene; (3) halogenated hydrocarbons such as methylene chloride, chloroform, and 1,2-dichloroethane; (4) ethers such as diethyl ether, tetrahydrofuran, t-butyl methyl ether, and 1,4-dioxane; (5) nitriles such as acetonitrile and propionitrile; (6) alcohols such as methanol, ethanol, n-propanol, and i-propanol; and (7) carboxylic acids such as acetic acid, propionic acid, and butyric acid. Of these, preferable examples are diethyl ether, tetrahydrofuran, t-butyl methyl ether, methanol, ethanol, and i-propanol. In particular, tetrahydrofuran, methanol, ethanol, and i-propanol are more preferable. It is possible to use a single solvent or a mixture of at least two of these.

The amount of the reaction solvent usable in the step (a) is not particularly limited. It may be at least one part by volume, preferably 1-50 parts by volume, more preferably 1-20 parts by volume, per one part by volume of the hydroxyl-protective mandelate of the formula 1.

The reaction of the step (a) may be conducted at a temperature of from −100° C. to +100° C., preferably from −80° C. to +80° C., more preferably from −60° C. to +60° C.

Although the reaction of the step (a) may terminate within 72 hr, the reaction time may vary depending on the types of the substrates used and the reaction conditions. Therefore, it is preferable to terminate the reaction after confirming that the raw material was almost completely consumed, by checking the progress of the reaction by a suitable analytical technique (e.g., gas chromatography, thin layer chromatography, liquid chromatography and NMR).

It is possible to obtain a crude product of the step (a) by conducting an ordinary post-treatment after the reaction. According to need, the crude product can be subjected to a purification such as the use of activated carbon, distillation, recrystallization, or column chromatography, thereby obtaining an optically active, hydroxyl-protective 2-hydroxy-1-(trifluoromethyl-substituted phenyl)ethanol of the formula 2 with high chemical purity.

The step (b), alkylation, is described in detail as follows. It is possible to conduct the step (b) by reacting the hydroxyl-protective hydroxyethanol of the formula 2 with an alkylation agent of the formula 3 (R²—X) in the presence of a base.

R² in the formula 3 represents a lower alkyl group having a carbon atom number of 1-6. It may be selected from methyl, ethyl, 1-propyl, 2-propyl, cyclopropyl, 1-butyl, 2-butyl, 2-methyl-1-propyl, t-butyl, cyclobutyl, 1-pentyl, 2-pentyl, 3-pentyl, neopentyl, t-amyl, cyclopentyl, 1-hexyl, 2-hexyl, 3-hexyl, cyclohexyl and the like.

The leaving group (represented by X) of the alkylation agent may be selected from chlorine, bromine, iodine, mesylate group (CH₃SO₂O), monochloromesylate group (CH₂ClSO₂O), tosylate group (p-MeC₆H₄SO₂O), triflate group (CF₃SO₂O) and the like. Of these, bromine, iodine, mesylate group, tosylate group, and triflate group are preferable, and bromine, iodine and mesylate group are more preferable.

The amount of the alkylation agent may be at least one equivalent, preferably 1-20 equivalents, more preferably 1-10 equivalents, per equivalent of the hydroxyl-protective hydroxyethanol of the formula 2.

The base used in the step (b) may be selected from (1) organic bases such as trimethylamine, triethylamine, diisopropylethylamine, tri-n-butylamine, dimethyllaurylamine, 4-N,N-dimethylaminopyridine, N,N-dimethylaniline, dimethylbenzylamine, 1,8-diazabicyclo[5.4.0]undec-7-ene, 1,4-diazabicyclo[2.2.2]octane, pyridine, 2,4-lutidine, 2,5-lutidine, 2,6-lutidine, 3,4-lutidine, 3,5-lutidine, 2,4,6-collidine, pyrimidine, and pyridazine; and (2) inorganic bases such as lithium hydride, sodium hydride, potassium hydride, calcium hydride, lithium carbonate, sodium carbonate, potassium carbonate, cesium carbonate, sodium hydrogencarbonate, potassium hydrogencarbonate, lithium hydroxide, sodium hydroxide, and potassium hydroxide. Of these, triethylamine, 4-N,N-dimethylaminopyridine, 1,8-diazabicyclo[5.4.0]undec-7-ene, 2,6-lutidine, sodium hydride, sodium carbonate, potassium carbonate, sodium hydrogencarbonate, and potassium hydrogencarbonate are preferable. In particular, triethylamine, 1,8-diazabicyclo[5.4.0]undec-7-ene, 2,6-lutidine, sodium hydride, sodium carbonate, and potassium carbonate are more preferable. These bases can be used alone or in combination.

The amount of the base may be at least one equivalent, preferably 1-20 equivalents, more preferably 1-10 equivalents, per equivalent of the hydroxyl-protective hydroxyethanol of the formula 2.

It may be possible to more smoothly conduct the alkylation by adding an additive. This additive may be selected from crown ethers (e.g., 12-crown-4,15-crown-5, and 18-crown-6), ethylene glycol dialkyl ethers (e.g., 1,2-dimethoxyethane, diethylene glycol dimethyl ether, and triethylene glycol dimethyl ether), and iodides (e.g., sodium iodide, potassium iodide, and tetrabutylammonium iodide). The additive to be used in the alkylation may be in an amount of at least 0.001 equivalents, preferably 0.001-50 equivalents, more preferably 0.001-20 equivalents, per equivalent of the hydroxyl-protective hydroxyethanol of the formula 2.

A reaction solvent usable in the step (b) is not particularly limited. Its examples are (1) aliphatic hydrocarbons such as n-pentane, n-hexane, cyclohexane, and n-heptane; (2) aromatic hydrocarbons such as benzene, toluene, ethylbenzene, xylene, and mesitylene; (3) halogenated hydrocarbons such as methylene chloride, chloroform, and 1,2-dichloroethane; (4) ethers such as diethyl ether, tetrahydrofuran, t-butyl methyl ether, and 1,4-dioxane; (5) esters such as ethyl acetate and n-butyl acetate; (6) amides such as hexamethylphosphoric triamide, N,N-dimethylformamide, N,N-dimethylacetamide, and N-methylpyrolidone. (7) nitriles such as acetonitrile and propionitrile; and (8) dimethylsulfoxide. Of these, preferable examples are toluene, 1,2-dichloroethane, tetrahydrofuran, ethyl acetate, N,N-dimethylformamide, N,N-dimethylacetamide, acetonitrile, and dimethylsulfoxide. In particular, more preferable examples are tetrahydrofuran, N,N-dimethylformamide, N,N-dimethylacetamide, acetonitrile, and dimethylsulfoxide. It is possible to use a single solvent or a mixture of at least two of these.

The amount of the reaction solvent usable in the step (b) is not particularly limited. It may be at least one part by volume, preferably 1-50 parts by volume, more preferably 1-20 parts by volume, per one part by volume of the hydroxyl-protective hydroxyethanol of the formula 2.

The reaction of the step (b) may be conducted at a temperature of from −50° C. to +200° C., preferably from −50° C. to +175° C., more preferably from −50° C. to +150° C.

Although the reaction of the step (b) may terminate within 72 hr, the reaction time may vary depending on the types of the substrates used and the reaction conditions. Therefore, it is preferable to terminate the reaction after confirming that the raw material was almost completely consumed, by checking the progress of the reaction by a suitable analytical technique (e.g., gas chromatography, thin layer chromatography, liquid chromatography and NMR).

It is possible to obtain a crude product of the step (b) by conducting an ordinary post-treatment after the reaction. According to need, the crude product can be subjected to a purification such as the use of activated carbon, distillation, recrystallization, or column chromatography, thereby obtaining an optically active, hydroxyl-protective 2-alkoxy-1-(trifluoromethyl-substituted phenyl)ethanol of the formula 4 with high chemical purity.

The step (c), deprotection, is described in detail as follows. It is possible to conduct the step (c) by deprotecting the hydroxyl-protective alkoxyethanol of the formula 4. In fact, the deprotection is a conversion of —OR¹ (R¹: a protecting group for hydroxyl group) into —OH. This protecting group can be classified into A-type, B-type, and C-type, depending on the way of the deprotection.

In case that a hydroxyl-protective alkoxyethanol of the formula 4 contains A-type protecting group, it is possible to conduct the step (c) by subjecting the hydroxyl-protective alkoxyethanol to a hydrolysis or solvolysis in the presence of an acid catalyst.

Examples of A-type protecting group include tetrahydropyranyl group, 1-ethoxyethyl group, methoxymethyl group, triphenylmethyl group, and trimethylsilyl group. Examples of the acid catalyst include organic acids (e.g., benzenesulfonic acid, p-toluenesulfonic acid, pyridinium p-toluenesulfonate (PPTS), SO₃H-type ion exchange resin, 10-camphorsulfonic acid, formic acid, acetic acid, trifluoroacetic acid, and trifluoromethanesulfonic acid) and inorganic acids (e.g., hydrochloric acid, hydrobromic acid, sulfuric acid, boric acid, and phosphoric acid). Of these, preferable examples are p-toluenesulfonic acid, hydrochloric acid, and sulfuric acid. In particular, more preferable examples are hydrochloric acid and sulfuric acid.

The acid catalyst may be used in an amount of 100 equivalents or less, preferably 0.01-50 equivalents, more preferably 0.01-25 equivalents, per equivalent of the hydroxyl-protective alkoxyethanol of the formula 4.

In case that a hydroxyl-protective alkoxyethanol of the formula 4 contains B-type protecting group, it is possible to conduct the step (c) by desilylation in the presence of fluorine ions. B-type protecting group can be defined as being a substituted silyl group, such as trimethylsilyl group, triethylsilyl group, t-butyldimethylsilyl group, and t-butyldiphenylsilyl group.

Examples of a fluorine-containing substance for generating the fluorine ions include tetrabutylammonium fluoride, a combination of hydrogen fluoride and triethylamine, a combination of hydrogen fluoride and pyridine, hydrofluoric acid, potassium fluoride, and cesium fluoride. Of these, preferable examples are tetrabutylammonium fluoride, HF-triethylamine, and hydrofluoric acid. In particular, more preferable examples are HF-triethylamine and hydrofluoric acid. The fluorine ions may be in an amount of 100 equivalents or less, preferably 0.01-50 equivalents, more preferably 0.01-25 equivalents, per equivalent of the hydroxyl-protective alkoxyethanol of the formula 4.

In case that a hydroxyl-protective alkoxyethanol of the formula 4 contains C-type protecting group, it is possible to conduct the step (c) by hydrogenolysis in the presence of a palladium catalyst. Examples of C-type protecting group include triphenylmethyl group and benzyl group.

Examples of the palladium catalyst include a combination of palladium and activated carbon, palladium hydroxide, palladium black, a combination of palladium and barium sulfate, a combination of palladium and alumina, and palladium sponge. Of these, preferable examples are Pd/activated carbon, palladium hydroxide, and Pd/alumina. In particular, more preferable examples are Pd/activated carbon and palladium hydroxide.

In case that palladium is loaded on a carrier (e.g., activated carbon) in the palladium catalyst, the content of such palladium may be 0.1-50 wt %, preferably 0.5-30 wt %, more preferably 1-20 wt %. In addition, in order to enhance safety during handling or to prevent oxidation of the palladium surface, it is possible to use one stored in water or mineral oil.

The palladium catalyst (in terms of metallic palladium) may be used in an amount of 20 wt % or less, preferably 0.001-15 wt %, more preferably 0.001-10 wt %, based on the total weight (100 wt %) of the hydroxyl-protective alkoxyethanol of the formula 4.

The above hydrogenolysis of the step (c) may be conducted by using hydrogen in an amount of at least one equivalent, per equivalent of the hydroxyl-protective alkoxyethanol of the formula 4. It is, however, usual to use hydrogen excessively due to the hydrogenolysis under a hydrogen atmosphere. The hydrogen pressure may be 5 MPa or less, preferably 0.01-3 MPa, more preferably 0.01-2 MPa. The hydrogen source for conducting the above hydrogenolysis may be formic acid, ammonium formate, hydrazine, and the like, besides molecular hydrogen.

The reaction solvent usable in the step (c) may be selected from (1) aliphatic hydrocarbons such as n-pentane, n-hexane, cyclohexane, and n-heptane; (2) aromatic hydrocarbons such as benzene, toluene, ethylbenzene, xylene, and mesitylene; (3) halogenated hydrocarbons such as methylene chloride, chloroform, and 1,2-dichloroethane; (4) ethers such as diethyl ether, tetrahydrofuran, t-butyl methyl ether, and 1,4-dioxane; (5) esters such as ethyl acetate and n-butyl acetate; (6) alcohols such as methanol, ethanol, n-propanol, and i-propanol; (7) carboxylic acids such as acetic acid, propionic acid, and butyric acid; (8) acidic aqueous solutions such as those of hydrochloric acid, sulfuric acid, hydrobromic acid, p-toluenesulfonic acid and 10-camphorsulfonic acid; and (9) water. Among these, toluene, ethyl acetate, methanol, ethanol, i-propanol, acetic acid and hydrochloric acid aqueous solution are preferable, while methanol, ethanol, i-propanol, acetic acid and hydrochloric acid aqueous solution are particularly more preferable. These reaction solvents can be used alone or in combination.

The amount of the reaction solvent usable in the step (c) is not particularly limited. It may be at least one part by volume, preferably 1-50 parts by volume, more preferably 1-20 parts by volume, per one volume of the hydroxyl-protective alkoxyethanol of the formula 4.

The step (c) may be conducted at a temperature of from −20° C. to +200° C., preferably from −20° C. to +150° C., more preferably from −20° C. to +100° C.

Although the reaction of the step (c) may terminate within 72 hr, the reaction time may vary depending on the types of the substrates used and the reaction conditions. Therefore, it is preferable to terminate the reaction after confirming that the raw material was almost completely consumed, by checking the progress of the reaction by a suitable analytical technique (e.g., gas chromatography, thin layer chromatography, liquid chromatography and NMR).

It is possible to obtain a crude product of the step (c) by conducting an ordinary post-treatment after the reaction. According to need, the crude product can be subjected to a purification such as the use of -activated carbon, distillation, recrystallization, or column chromatography, thereby obtaining the target product, an optically active 2-alkoxy-1-(trifluoromethyl-substituted phenyl)ethanol derivative of the formula 5 with high chemical purity.

The following nonlimitative example is illustrative of the present invention.

### EXAMPLE

### Step (d), Esterification

3.46 g (15.72 mmol, 1 eq.) of optically active (S)-4-trifluoromethylmandelic acid (having 98% ee) and 0.09 g (0.92 mmol, 0.06 eq.) of concentrated sulfuric acid were added to 7.9 ml of methanol. The resulting mixture was stirred for 7 hr under a reflux condition. After the reaction, saturated brine was added to the reaction liquid, followed by extraction with ethyl acetate. The recovered organic layer was washed with a saturated sodium hydrogencarbonate aqueous solution and then with saturated brine, followed by drying with anhydrous sodium sulfate, filtration, concentration and vacuum drying, thereby obtaining 3.19 g of a crude product of optically active methyl-(S)-4-trifluoromethylmandelate represented by the following formula. The yield was 87%.

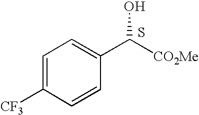

NMR data are as follows.

¹H-NMR (standard substance: TMS; solvent: CDCl₃), δppm: 3.79 (s, 3H), 5.25 (s, 1H), 7.57 (d, Ar—H, 2H), 7.63 (d, Ar—H, 2H).

### Step (e), Hydroxyl Group Protection

To 13.6 ml of methylene chloride, there were added 3.19 g (13.62 mmol, 1 eq.) of the above-obtained crude product of optically active methyl-(S)-4-trifluoromethylmandelate, 1.72 g (20.45 mmol, 1.50 eq.) of DHP, and 0.04 g (0.16 mmol, 0.01 eq.) of PPTS, followed by stirring at room temperature for 18 hr. After the reaction, a saturated sodium hydrogencarbonate aqueous solution was added to the reaction liquid, followed by extraction with ethyl acetate. The recovered organic layer was washed with saturated brine, followed by drying with anhydrous sodium sulfate, filtration, concentration and vacuum drying, thereby obtaining 4.33 g of a crude product of optically active, THP-protective methyl-(S)-4-trifluoromethylmandelate represented by the following formula. The yield was 100%.

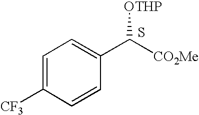

NMR data are as follows.

¹H-NMR (standard substance: TMS; solvent: CDCl₃), δppm: 1.40-2.05 (m, 6H), 3.40-3.60 (m, 1H), 3.60-3.70 (m, 0.5H), 3.73 (s, 3H), 3.85-4.00 (m, 0.5H), 4.58 (t, 0.5H), 4.91 (t, 0.5H), 5.29 (s, 0.5H), 5.39 (s, 0.5H), 7.56-7.67 (d, d, s, Ar—H, 4H).

### Step (a), Hydride Reduction

4.33 g (13.60 mmol, 1 eq.) of the crude product of optically active, THP-protective methyl-(S)-4-trifluoromethylmandelate obtained by the step (e) were added to 13.6 ml of methanol, followed by cooling to 0° C. Then, 1.01 g (26.70 mmol, 1.96 eq.) of sodium borohydride were added, followed by stirring at the same temperature for 1 hr and then at room temperature for 3 hr. After the reaction, water was added to the reaction liquid to decompose the remaining sodium borohydride, followed by extraction with ethyl acetate. The recovered organic layer was washed with saturated brine, followed by drying with anhydrous sodium sulfate, filtration, concentration and vacuum drying, thereby obtaining 3.85 g of a crude product of optically active, THP -protective (S)-2-hydroxy-1- (4′-trifluoromethylphenyl) ethanol represented by the following formula. The yield was 97%.

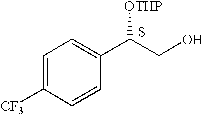

NMR data are as follows.

¹H-NMR (standard substance: TMS; solvent: CDCl₃), δ ppm: 1.35-2.00 (m, 6H), 3.10 (br, 1H), 3.30 (dt, 0.5H), 3.54 (dd, 0.5H), 3.57 (dd, 0.5H), 3.62-3.80 (m, 2H), 4.05 (dt, 0.5H), 4.55 (dd, 0.5H), 4.79 (dd, 0.5H), 4.88 (t, 0.5H), 4.91 (t, 0.5H), 7.45 (d, Ar—H, 1H), 7.51 (d, Ar—H, 1H), 7.61 (d, Ar—H, 2H).

### Step (b), Alkylation

3.85 g (13.26 mmol, 1 eq.) of the crude product of optically active, THP-protective (S)-2-hydroxy-1-(4′-trifluoromethylphenyl)ethanol obtained by the step (a) were added to 13.3 ml of tetrahydrofuran, followed by cooling to 0° C. Then, 0.82 g (20.50 mmol, 1.55 eq.) of 60% sodium hydride were added, followed by addition of 2.81 g (19.80 mmol, 1.49 eq.) of methyl iodide and then stirring at the same temperature for 10 min and then at room temperature for 30 min. After the reaction, water was added to the reaction liquid to decompose the remaining sodium hydride, followed by extraction with ethyl acetate. The recovered organic layer was washed with saturated brine, followed by drying with anhydrous sodium sulfate, filtration, concentration and vacuum drying, thereby obtaining 4.09 g of a crude product of optically active, THP-protective (S)-2-methoxy-1-(4′-trifluoromethylphenyl)ethanol represented by the following formula. The yield was quantitative.

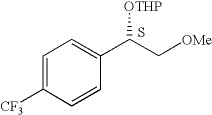

NMR data are as follows.

¹H-NMR (standard substance: TMS; solvent: CDCl₃), δppm: 1.30-2.00 (m, 6H), 3.28-3.40 (m, 0.5H), 3.37 (s, 1.5H), 3.39 (s, 1.5H), 3.45-3.58 (m, 2H), 3.60 (dd, 0.5H), 3.67 (dd, 0.5H), 3.95-4.10 (m, 0.5H), 4.43 (t, 0.5H), 4.88 (dd, 0.5H), 4.90-5.05 (m, 1H), 7.46 (d, Ar—H, 1H), 7.53 (d, Ar—H, 1H), 7.60 (dd, Ar—H, 2H).

### Step (c), Hydroxyl Group Deprotection

To 50.0 ml of methanol, there were added 4.09 g (13.44 mmol, 1 eq.) of the crude product of optically active, THP-protective (S)-2-methoxy-1-(4′-trifluoromethylphenyl)ethanol obtained by the step (b) and 4.80 g (48.71 mmol, 3.62 eq.) of 37% hydrochloric acid, followed by stirring at room temperature for 26 hr. After the reaction, the reaction liquid was concentrated, followed by addition of water and then extraction with ethyl acetate. The recovered organic layer was washed with a saturated sodium hydrogencarbonate aqueous solution and then with saturated brine, followed by drying with anhydrous sodium sulfate, filtration, concentration and vacuum drying, thereby obtaining 3.05 g of a crude product of optically active (S)-2-methoxy-1-(4′-trifluoromethylphenyl)ethanol represented by the following formula. The yield was quantitative. The total yield from optically active (S)-4-trifluoromethylmandelic acid of the step (d) was 84%. The product of the step (c) was found by chiral gas chromatography to have an optical purity of 94% ee. Furthermore, it was dextrorotatory (+) with respect to rotatory polarization.

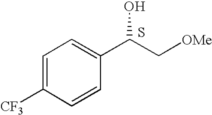

NMR data of the product of the step (c) are as follows.

¹H-NMR (standard substance: TMS; solvent: CDCl₃), δppm: 2.88 (br, 1H), 3.41 (dd, 1H), 3.44 (s, 3H), 3.57 (dd, 1H), 4.95 (dd, 1H), 7.51 (d, Ar—H, 2H), 7.61 (d, Ar—H, 2H).

The entire contents of Japanese Patent Application No. 2002-310180 (filed Oct. 24, 2002), which is a basic Japanese application of the present application, are incorporated herein by reference.

1. A process for producing an optically active 2-alkoxy-1-(trifluoromethyl-substituted phenyl)ethanol derivative represented by the formula 5: 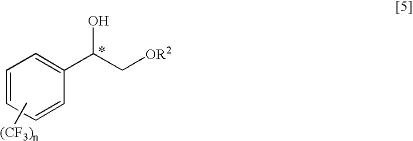
where R² represents a lower alkyl group having a carbon atom number of 1-6, n represents an integer of 1 or 2, and * represents a chiral carbon, the process comprising the steps of:
(a) reducing an optically active, hydroxyl-protective, trifluoromethyl-substituted mandelate represented by the formula 1, by a hydride reducing agent, 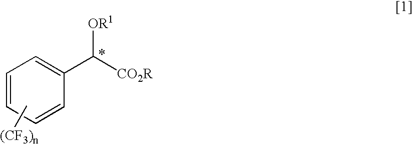
where R represents a lower alkyl group having a carbon atom number of 1-6, R¹ represents a protecting group for hydroxyl group, and n and * are defined as in the formula 5, into an optically active, hydroxyl-protective 2-hydroxy-1-(trifluoromethyl-substituted phenyl)ethanol represented by the formula 2: 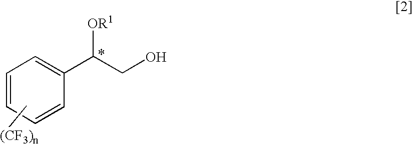
where R¹ is defined as in the formula 1, and n and * are defined as in the formula 5,
(b) reacting the hydroxyl-protective hydroxyethanol represented by the formula 2, with an alkylation agent represented by the formula 3, in the presence of a base,
R²—X [3]
where R² is defined as in the formula 5, and X represents a leaving group, thereby producing an optically active, hydroxyl-protective 2-alkoxy-1-(trifluoromethyl-substituted phenyl)ethanol represented by the formula 4: 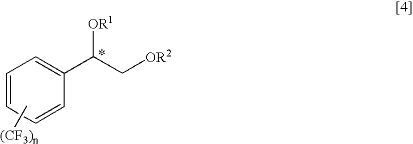
where R¹ is defined as in the formula 1, and R², n and * are defined as in the formula 5, and
(c) deprotecting the hydroxyl-protective alkoxyethanol represented by the formula 4 into the alkoxyethanol derivative represented by the formula 5.

2. A process for producing an optically active 2-methoxy-1-(4′-trifluoromethylphenyl)ethanol represented by the formula 10: 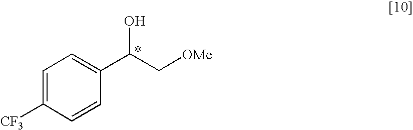
where Me represents a methyl group and * represents a chiral carbon, the process comprising the steps of:
(a) reducing an optically active, hydroxyl-protective 4-trifluoromethylmandelate represented by the formula 6, by sodium borohydride, 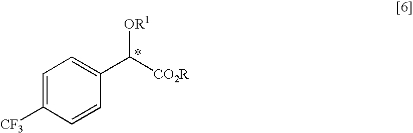
where R represents a lower alkyl group having a carbon atom number of 1-6, R¹ represents a protecting group for hydroxyl group, and * is defined as in the formula 10, into an optically active, hydroxyl-protective 2-hydroxy-1-(4′-trifluoromethylphenyl)ethanol represented by the formula 7: 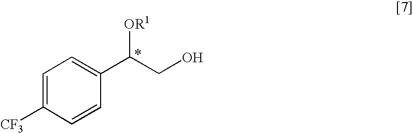
where R¹ is defined as in the formula 6, and * is defined as in the formula 10,
(b) reacting the hydroxyl-protective hydroxyethanol represented by the formula 7, with a methylation agent represented by the formula 8, in the presence of a base,
Me-X [8]
where Me represents a methyl group, and X represents a leaving group, thereby producing an optically active, hydroxyl-protective 2-methoxy-1-(4′-trifluoromethylphenyl)ethanol represented by the formula 9: 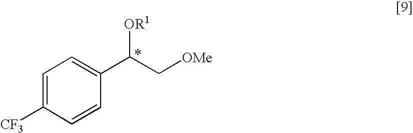
where R¹ is defined as in the formula 6, and Me and * are defined as in the formula 10, and
(c) deprotecting the hydroxyl-protective methoxyethanol represented by the formula 9 into the methoxyethanol represented by the formula 10.

3. A process according to claim 1, wherein the optically active, hydroxyl-protective, trifluoromethyl-substituted mandelate represented by the formula 1 is produced by a process comprising the steps of:
(d) reacting an optically active trifluoromethyl-substituted mandelic acid represented by the formula 11, with a lower alcohol having a carbon atom number of 1-6 in the presence of an acid catalyst, 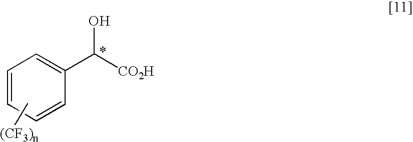
where n and * are defined as in the formula 5, thereby producing an optically active trifluoromethyl-substituted mandelate represented by the formula 12: 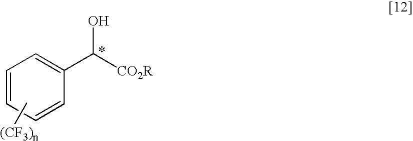
where R is defined as the formula 1, and n and * are defined as in the formula 5, and
(e) protecting a hydroxyl group of the mandelate represented by the formula 12, thereby producing the hydroxyl-protective mandelate represented by the formula 1.

4. A process according to claim 2, wherein the optically active, hydroxyl-protective 4-trifluoromethylmandelate represented by the formula 6 is produced by a process comprising the steps of:
(d) reacting an optically active 4-trifluoromethylmandelic acid represented by the formula 13, with a lower alcohol having a carbon atom number of 1-6 in the presence of an acid catalyst, 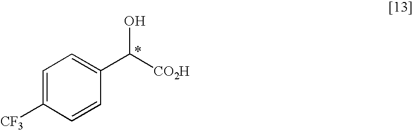
where * is defined as in the formula 10, thereby producing an optically active 4-trifluoromethylmandelate represented by the formula 14: 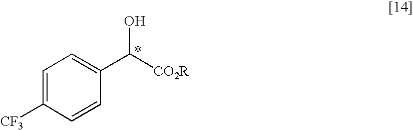
where R is defined as the formula 6, and * is defined as in the formula 10, and
(e) protecting a hydroxyl group of the mandelate represented by the formula 14, thereby producing the hydroxyl-protective mandelate represented by the formula 6.

5. A process according to claim 1, wherein the hydride reducing agent of the step (a) is selected from the group consisting of LiAlH₄, diborane, NaBH₄, and LiBH₄.

6. A process according to claim 5, wherein the hydride reducing agent of the step (a) is NaBH₄.

7. A process according to claim 1, wherein the step (a) is conducted in a reaction solvent that is at least one selected from the group consisting of tetrahydrofuran, methanol, ethanol, and i-propanol.

8. A process according to claim 1, wherein X of the formula 3 is selected from the group consisting of bromine, iodine, mesylate group, tosylate group, and triflate group.

9. A process according to claim 8, wherein X of the formula 3 is selected from the group consisting of bromine, iodine, and mesylate group.

10. A process according to claim 1, wherein the base of the step (b) is at least one selected from the group consisting of triethylamine, 4-N,N-dimethylaminopyridine, 1,8-diazabicyclo[5.4.0]undec-7-ene, 2,6-lutidine, sodium hydride, sodium carbonate, potassium carbonate, sodium hydrogencarbonate, and potassium hydrogencarbonate.

11. A process according to claim 10, wherein the base of the step (b) is at least one selected from the group consisting of triethylamine, 1,8-diazabicyclo[5.4.0]undec-7-ene, 2,6-lutidine, sodium hydride, sodium carbonate, and potassium carbonate.

12. A process according to claim 1, wherein the step (b) is conducted in a reaction solvent that is at least one selected from the group consisting of tetrahydrofuran, N,N-dimethylformamide, N,N-dimethylacetamide, acetonitrile, and dimethylsulfoxide.

13. A process according to claim 1, wherein the step (c) is conducted by a hydrolysis or solvolysis in the presence of an acid catalyst.

14. A process according to claim 13, wherein R¹ of the formula 4 is selected from the group consisting of tetrahydropyranyl group, 1-ethoxyethyl group, methoxymethyl group, triphenylmethyl group, and trimethylsilyl group.

15. A process according to claim 13, wherein the acid catalyst is selected from the group consisting of p-toluenesulfonic acid, hydrochloric acid, and sulfuric acid.

16. A process according to claim 1, wherein R¹ of the formula 4 or 9 is a substituted silyl group, and
wherein the step (c) is conducted by a desilylation in the presence of fluorine ions.

17. A process according to claim 16, wherein the substituted silyl group is selected from the group consisting of trimethylsilyl group, triethylsilyl group, t-butyldimethylsilyl group, and t-butyldiphenylsilyl group.

18. A process according to claim 16, wherein the fluorine ions originate from a fluorine-containing substance selected from the group consisting of tetrabutylammonium fluoride, a combination of hydrogen fluoride and triethylamine, and hydrofluoric acid.

19. A process according to claim 1, wherein the step (c) is conducted by a hydrogenolysis in the presence of a palladium catalyst.

20. A process according to claim 19, wherein R¹ of the formula 4 is a triphenylmethyl group or benzyl group.

21. A process according to claim 19, wherein the palladium catalyst is selected from the group consisting of a combination of palladium and activated carbon, palladium hydroxide, and a combination of palladium and alumina.

22. A process according to claim 1, wherein the step (c) is conducted in a reaction solvent that is at least one selected from the group consisting of methanol, ethanol, i-propanol, acetic acid, and a hydrochloric acid aqueous solution.

23. A process according to claim 3, wherein the lower alcohol of the step (d) is methanol.

24. A process according to claim 3, wherein the acid catalyst of the step (d) is selected from the group consisting of p-toluenesulfonic acid, sulfuric acid, and zinc chloride.

25. A process according to claim 24, wherein the acid catalyst of the step (d) is sulfuric acid.

26. A process according to claim 3, wherein the step (e) is conducted by reacting the mandelate, represented by the formula 12, with a protecting agent in the presence of an acid catalyst.

27. A process according to claim 26, wherein the protecting agent is dihydropyrane or ethyl vinyl ether, and
wherein the acid catalyst is p-toluenesulfonic acid or pyridinium p-toluenesulfonate.

28. A process according to claim 3, wherein the step (e) is conducted by reacting the mandelate, represented by the formula 12, with a protecting agent in the presence of a base.

29. A process according to claim 28, wherein the protecting agent is selected from the group consisting of methoxymethyl chloride, triphenylmethyl chloride, benzyl bromide, trimethylsilyl chloride, triethylsilyl chloride, t-butyldimethylsilyl chloride, and t-butyldiphenylsilyl chloride, and
wherein the base is selected from the group consisting of sodium hydride, triethylamine, 4-N,N-dimethylaminopyridine, and imidazole.

30. A process according to claim 3, wherein the step (e) is conducted in a reaction solvent that is at least one selected from the group consisting of toluene, methylene chloride, tetrahydrofuran, ethyl acetate, and N,N-dimethylformamide.

31. An optically active, hydroxyl-protective, trifluoromethyl-substituted mandelate represented by the formula 1, 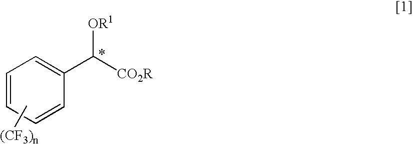
where R represents a lower alkyl group having a carbon atom number of 1-6, R¹ represents a protecting group for hydroxyl group, n represents an integer of 1 or 2, and * represents a chiral carbon.

32. An optically active, hydroxyl-protective 4-trifluoromethylmandelate represented by the formula 6, 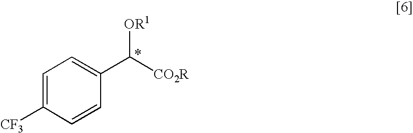
where R represents a lower alkyl group having a carbon atom number of 1-6, R¹ represents a protecting group for hydroxyl group, and * represents a chiral carbon.

33. An optically active, hydroxyl-protective, 2-hydroxy-1-(trifluoromethyl-substituted phenyl)ethanol represented by the formula 2: 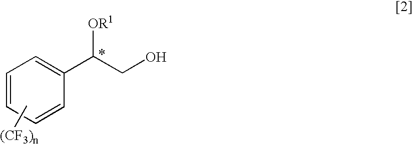
where R¹ represents a protecting group for hydroxyl group, n represents an integer of 1 or 2, and * represents a chiral carbon.

34. An optically active, hydroxyl-protective 2-hydroxy-1-(4′-trifluoromethylphenyl)ethanol represented by the formula 7: 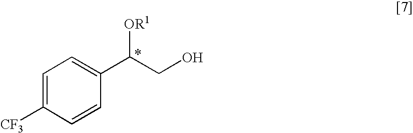
where R¹ represents a protecting group for hydroxyl group, and * represents a chiral carbon.

35. An optically active, hydroxyl-protective 2-alkoxy-1-(trifluoromethyl-substituted phenyl)ethanol represented by the formula 4: 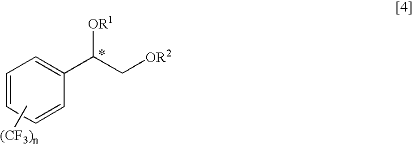
where R¹ represents a protecting group for hydroxyl group, R² represents a lower alkyl group having a carbon atom number of 1-6, n represents an integer of 1 or 2, and * represents a chiral carbon.

36. An optically active, hydroxyl-protective 2-methoxy-1-(4′-trifluoromethylphenyl)ethanol represented by the formula 9: 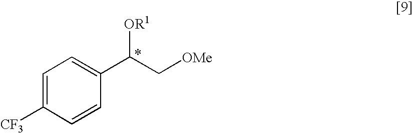
where R¹ represents a protecting group for hydroxyl group, Me represents a methyl group, and * represents a chiral carbon.

37. An optically active 2-alkoxy-1-(trifluoromethyl-substituted phenyl)ethanol derivative represented by the formula 5: 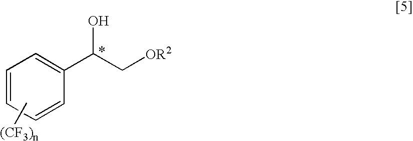
where R² represents a lower alkyl group having a carbon atom number of 1-6, n represents an integer of 1 or 2, and * represents a chiral carbon.

38. An optically active 2-methoxy-1-(4′-trifluoromethylphenyl)ethanol represented by the formula 10: 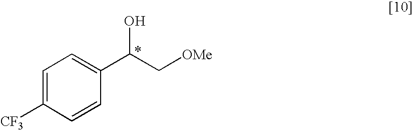
where Me represents a methyl group and * represents a chiral carbon.

39. An optically active trifluoromethyl-substituted mandelic acid represented by the formula 11, 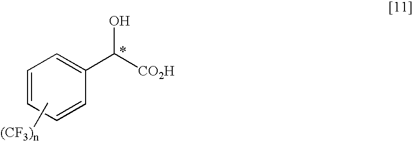
where n represents an integer of 1 or 2, and * represents a chiral carbon;
where optically active 4-trifluoromethylmandelic acid, (R)-3-trifluoromethylmandelic acid, and optically active 3,5-bis(trifluoromethyl)mandelic acid are excluded from the mandelic acid represented by the formula 11.

40. An optically active trifluoromethyl-substituted mandelate represented by the formula 12: 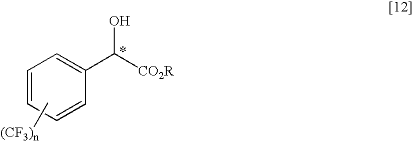
where R represents a lower alkyl group having a carbon atom number of 1-6, n represents an integer of 1 or 2, and * represents a chiral carbon;
where ethyl-(R)-4-trifluoromethylmandelate is excluded from the mandelate represented by the formula 12.

41. An optically active methyl trifluoromethyl-substituted mandelate represented by the formula 15: 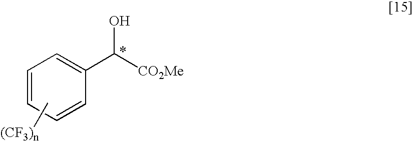
where Me represents a methyl group, n represents an integer of 1 or 2, and * represents a chiral carbon.

42. An optically active 4-trifluoromethylmandelate represented by the formula 14: 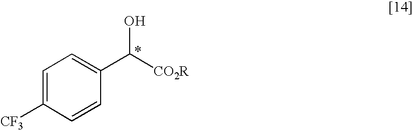
where R represents a lower alkyl group having a carbon atom number of 1-6, and * represents a chiral carbon;
where ethyl-(R)-4-trifluoromethylmandelate is excluded from the mandelate represented by the formula 14.

43. An optically active methyl-4-trifluoromethylmandelate represented by the formula 16: 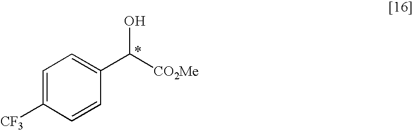
where Me represents a methyl group, and * represents a chiral carbon.
